(19) United States
(12) Patent Application Publication
(10) Pub. No.: US 2013/0310569 A1
(43) Pub. Date: Nov. 21, 2013

(54) TRIAZOLIDE BASED IONIC LIQUIDS

(71) Applicants: Hunaid Nulwala, Pittsburgh, PA (US); Robert Thompson, Pittsburgh, PA (US); David Luebke, Bethel Park, PA (US); Erik Albenze, Bethel Park, PA (US)

(72) Inventors: Hunaid Nulwala, Pittsburgh, PA (US); Robert Thompson, Pittsburgh, PA (US); David Luebke, Bethel Park, PA (US); Erik Albenze, Bethel Park, PA (US)

(21) Appl. No.: 13/898,843

(22) Filed: May 21, 2013

Related U.S. Application Data
(60) Provisional application No. 61/688,778, filed on May 21, 2012.

(51) Int. Cl. C07D 249/06; C07D 249/04; C07F 7/10

(52) U.S. Cl. CPC C07D 249/06; C07F 7/10; C07D 249/04
USPC 548/110; 548/255

(57) ABSTRACT

A method of synthesizing an ionic liquid, includes reacting a 1,2,3-triazole including at least one of a 4-substituent or a 5-substituent with a hydroxide compound having the formula R⁺OH⁻ in a dehydration reaction, wherein R⁺ is an ionic liquid cation. R⁺ is a five-membered heterocyclic cation, an aromatic cation, a sulfonium cation, an ammonium cation, or a phosphonium cation. In a number of embodiments, R⁺ is a pyridinium cation, a bipyridinium cation, an amino pyridinium cation, a pyridazinium cation, an ozaxolium cation, a pyrazolium cation, an imidazolium cation, a pyramidinium cation, a triazolium cation, a thiazolium cation, an acridinium cation, a quinolinium cation, an isoquinolinium cation, an orange-acridinium cation, a benzotriazolium cation, a methimzolium cation, a sulfonium cation, an ammonium cation, or a phosphonium cation.

### CROSS-REFERENCE TO RELATED APPLICATIONS

This application claims benefit of U.S. Provisional Patent Application Ser. No. 61/688,778, filed May 21, 2012, the disclosure of which is incorporated herein by reference.

### GOVERNMENTAL INTEREST

This invention was made with government support under grant no. DE-FE0004000 awarded by the Department of Energy, National Energy Technology Laboratory. The government has certain rights in this invention.

### BACKGROUND

The following information is provided to assist the reader in understanding technologies disclosed below and the environment in which such technologies may typically be used. The terms used herein are not intended to be limited to any particular narrow interpretation unless clearly stated otherwise in this document. References set forth herein may facilitate understanding of the technologies or the background thereof. The disclosure of all references cited herein are incorporated by reference.

Ionic liquids (ILs) are organic salts which are liquids at temperatures below, for example, 100° C. Many ILs are liquids at room temperature (that is, approximately 25° C.). ILs possess attractive properties including, for example, unique solubilities, wide electrochemical windows, negligible vapor pressures, good thermal stabilities, low flammability and a potentially environmentally benign nature. Such properties have resulted in use of ILs in various applications including use as electrolytes, solvents, catalysts, and in gas separations. ILs also have a high carbon dioxide (CO₂) solubility, making them a good media for CO₂ separation in solvent and membrane processes.

To, for example, address the issue of rising levels of CO₂ in the atmosphere, more effective technologies are required for its economical capture and storage. Power generation based on fossil fuels offers an opportunity to capture large quantities of CO₂ at centralized locations. There have been a number of ongoing efforts to develop technologies to capture CO₂ from such sources. Existing CO₂ capture systems are based either on chemical or physical absorption. Chemical absorption systems, commonly used in post-combustion capture applications, use reactive solvents such as monoethanolamine (MEA) in combination with heat-induced CO₂ recovery. Physical absorption systems are more common in pre-combustion capture systems, and they employ ether-based solvents such as polyethylene glycol dimethyl ether (SELEXOL®, a commercial solvent for removal of acid gas from gasses, available from Union Carbide Corporation of New York, N.Y.) in combination with pressure-induced CO₂ recovery.

Real-world CO₂ gas streams have significant concentrations of water, and the cost of removing moisture from currently used in pre-combustion carbon capture solvents such as SELEXOL raises the cost of this process to unacceptably high levels. Design of any carbon capture solvent should account for how water affects the CO₂ absorption process.

Although numerous properties make ILs effective for the chemisorption of CO₂, they tend to suffer from high viscosities which can raise the capital costs of CO₂ capture systems and render them inefficient. In case of CO₂-reactive ILs, the presence of hydrogen atoms available for hydrogen bonding in the anions of ILs has been proposed to lead to high viscosities through the formation of salt-bridges between the anions.

### SUMMARY

In one aspect, a method of synthesizing an ionic liquid includes reacting a 1,2,3-triazole comprising at least one of a 4-substituent or a 5-substituent with a hydroxide compound having the formula R⁺OH⁻ in a dehydration reaction, wherein R⁺ is an ionic liquid cation. R⁺ may, for example, be a five-membered heterocyclic cation, an aromatic cation, a sulfonium cation, an ammonium cation, or a phosphonium cation. In a number of embodiments, R⁺ is a pyridinium cation, a bipyridinium cation, an amino pyridinium cation, a pyridazinium cation, an ozaxolium cation, a pyrazolium cation, an imidazolium cation, a pyramidinium cation, a triazolium cation, a thiazolium cation, an acridinium cation, a quinolinium cation, an isoquinolinium cation, an orange-acridinium cation, a benzotriazolium cation, a methimzolium cation, a sulfonium cation, an ammonium cation, or a phosphonium cation. R⁺ may, for example, be a cation having the formula:

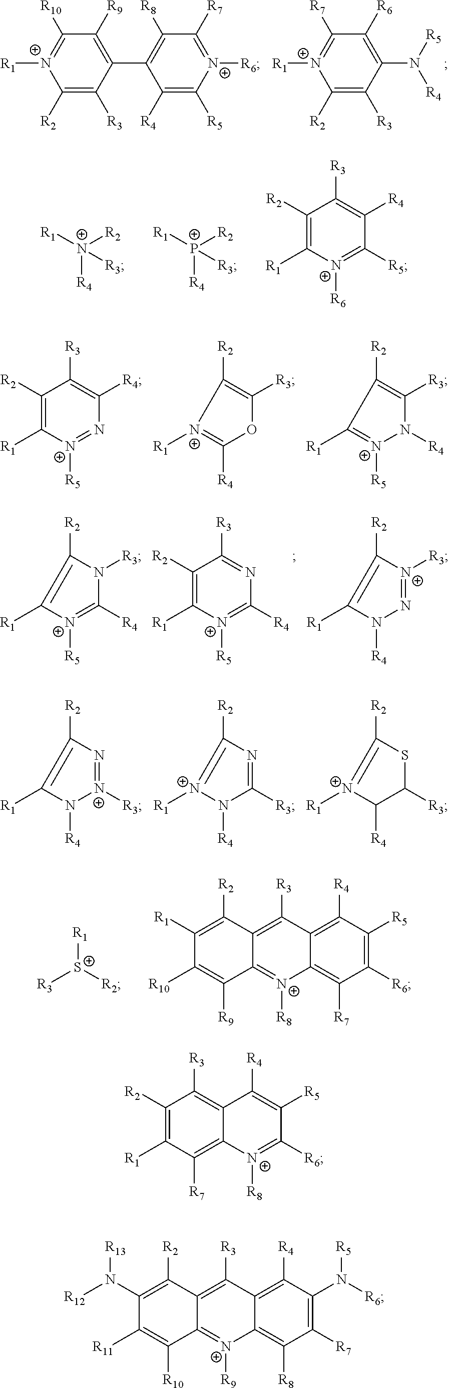

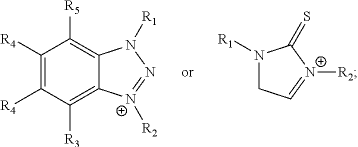

wherein R¹, R¹³ are independently, the same or different, a C₁-C₆ alkyl group, a C₁-C₆ alkenyl group, C₁-C₆ hydroxyalkyl group, a C₁-C₆ haloalkyl group, a C₂-C₈ alkoxylalkyl group, a C₆-C₁₀ aryl group a C₈-C₁₆ arylalkyl group or a C₈-C₁₆ alkylaryl group. In a number of embodiments, R⁺ is an imidazolium cation, an ammonium cation, or a phosphonium cation. In a number of embodiments, R⁺ is a phosphonium cation.

The 1,2,3-triazole including at least one of a 4-substituent or a 5-substituent may, for example, have the formula:

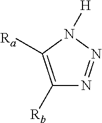

wherein Rₐ and R_{b} are independently H, an electron withdrawing group, an electron donating group, or a hydrophobic group and at least one of Rₐ, and R_{b} is not H. In a number of embodiments, Rₐ, and R_{b} are independently, the same or different, H, a halo group, a C₁-C₈ alkyl group, a C₂-C₈ alkenyl group, C₂-C₈ alkynyl group, —Si(R_{d}RₑR_{f}), —(R_{g})Si(R_{d}RₑR_{f}), wherein R_{g} is a C₂-C₆ alkylene group, and R_{d}, Rₑ and R_{f} are independently a C₁-C₈ alkyl group or a C₁-C₁₀ aryl group, —(Rₕ)ORᵢ wherein Rₕ is a C₂-C₆ alkylene group and Rᵢ is a C₁-C₈ alkyl group, a C₁-C₈ haloalkyl, a C₂-C₈ alkenyl group, a phenyl group or an C₂-C₈ alkynyl group, —ORᵢ, a cyano group, a C₁-C₆ cyanoalkyl group, a C₁-C₈ hydroxyalkyl group, —(Rⱼ)(O)ORₖ, wherein Rⱼ is an C₂-C₆ alkylene group and Rₖ is a C₁-C₈ alkyl group, a C₁-C₈ haloalkyl group, a C₂-C₈ alkenyl group or a C₂-C₈ alkynyl group, an amine group, a C₁-C₈ alkylamine, a C₁-C₈ dialkylamine group, a C₁-C₈ haloalkyl group, a C₆-C₁₀ aryl group, a C₈-C₁₆ alkylaryl group, a C₈-C₁₆ arylalkyl group, —(R₁₅)ₙ[Si(Rₗ)(Rₘ)O]ₚSi(R_{q}RᵣRₛ), wherein n is an integer from 1 to 5, p is an integer from 1 to 9, wherein R₁₅ is a C₂-C₃ alkylene group, and Rₗ, Rₘ, R_{q}, Rᵣ and Rₛ are independently, the same or different, H, a C₁-C₈ alkyl group (or a C₁-C₄ alkyl group in some embodiments) or a phenyl group, or —R₁₆O[C(R₁₇)(R₁₈)]ᵤO[C(R₁₉)(R₂₀)]ᵥO[C(R₂₁)(R₂₂)]ₓOR₂₃, wherein R₁₆ is a C₂-C₆ alkylene group, R₁₇, R₁₈, R₁₉, R₂₀, R₂₁, R₂₂ and R₂₃ are independently, the same or different, H, a C₁-C₈ alkyl group (or a C₁-C₄ alkyl group in some embodiments) or a phenyl group, wherein u is an integer of 2 or 3, v is an integer of 0, 2 or 3 and x is an integer of 0, 2 or 3, and wherein at least one of Rₐ and Rₕ is not H. In a number of embodiments, neither Rₐ, nor R_{b} is H. In a number of embodiments, Rₐ, and R_{b} are independently H, Cl, F, a C₁-C₆ alkyl group, —CF₃, —COOCH₃, —CH₂OH, —CH₂OCH₃, —C≡N, —CH₂≡N, —C≡CH, —CH₂C≡CH, —CHC═CH₂, a phenyl group. In a number of other embodiments, Rₐ, and R_{b} are independently H or a C₁-C₃ alkyl group

The 1,2,3-triazole including at least one of a 4-substituent or a 5-substituent may, for example, be synthesized by reacting an alkyne having the formula:

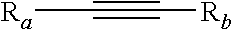

with an azide having the formula R_{c}—N₃ in a 3+2 cycloaddition reaction, wherein R_{c} is a labile amino protecting group. The labile amino protecting group may, for example, be a carbobenzyloxy group, a p-methoxybenzyl carbonyl group, a tert-butyloxycarbonyl group, a 9-fluorenylmethyloxycarbonyl group, an acetyl group, a benzoyl group, a benzyl group, a carbamate group, a p-methoxybenzyl group, a 3,4-dimethoxybenzyl group, a p-methoxyphenyl group, a tosyl group, a mesyl group, a dimethoxytrityl group, a [bis-(4-methoxyphenyl)phenylmethyl] group, a pivaloyl group, a trimethylsilyl group, a tert-butyldimethylsilyl group, a tri-iso-propylsilyloxymethyl group, and a triisopropylsilyl group. After reaction of the alkine with the azide, the labile amino protecting group is removed.

In another aspect, an ionic liquid includes a mono- or di-substituted 1,2,3-triazole and an ionic liquid cation. The ionic liquid may, for example, have the formula:

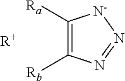

wherein R⁺ is s a five-membered heterocyclic cation, an aromatic cation, a sulfonium cation, an ammonium cation, or a phosphonium cation as set forth above and R^{a} and R^{b} are defined as set forth above. As also described above, R⁺ may, for example, be a pyridinium cation, a bipyridinium cation, an amino pyridinium cation, a pyridazinium cation, an ozaxolium cation, a pyrazolium cation, an imidazolium cation, a pyramidinium cation, a triazolium cation, a thiazolium cation, an acridinium cation, a quinolinium cation, an isoquinolinium cation, an orange-acridinium cation, a benzotriazolium cation, a methimzolium cation, a sulfonium cation, an ammonium cation, or a phosphonium cation.

In another aspect, a compound has the formula:

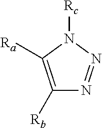

wherein Rₐ, and R_{b} are independently, the same or different, H, a halo group, a C₁-C₈ alkyl group, a C₂-C₈ alkenyl group, C₂-C₈ alkynyl group, —Si(R_{d}RₑR_{f}), —(R_{g})Si(R_{d}RₑR_{f}), wherein R_{g} is a C₂-C₆ alkylene group, and R_{d}, Rₑ and R_{f} are independently a C₁-C₈ alkyl group or a C₁-C₁₀ aryl group, —(Rₕ)ORᵢ wherein Rₕ is a C₂-C₆ alkylene group and Rᵢ is a C₁-C₈ alkyl group, a C₁-C₈ haloalkyl, a C₂-C₈ alkenyl group, a phenyl group or an C₂-C₈ alkynyl group, —ORᵢ, a cyano group, a C₁-C₆ cyanoalkyl group, a C₁-C₈ hydroxyalkyl group, —(Rⱼ)(O)ORₖ, wherein Rⱼ is an C₂-C₆ alkylene group and Rₖ is a C₁-C₈ alkyl group, a C₁-C₈ haloalkyl group, a C₂-C₈ alkenyl group or a C₂-C₈ alkynyl group, an amine group, a C₁-C₈ alkylamine, a C₁-C₈ dialkylamine group, a C₁-C₈ haloalkyl group, a C₆-C₁₀ aryl group, a C₈-C₁₆ alkylaryl group, a C₈-C₁₆ arylalkyl group, —(R₁₅)ₙ[Si(Rₗ)(Rₘ)O]ₚSi(R_{q}RᵣRₛ), wherein n is an integer from 1 to 5, p is an integer from 1 to 9, wherein R₁₅ is a C₂-C₃ alkylene group, and Rₗ, Rₘ, R_{q}, Rᵣ and Rₛ are independently, the same or different, H, a C₁-C₈ alkyl group (or a C₁-C₄ alkyl group in some embodiments) or a phenyl group, or —R₁₆O[C(R₁₇)(R₁₈)]ᵤO[C(R₁₉)(R₂₀)]ᵥO[C(R₂₁)(R₂₂)]ₓOR₂₃, wherein R₁₆ is a C₂-C₆ alkylene group, R₁₇, R₁₈, R₁₉, R₂₀ R₂₁, R₂₂ and R₂₃ are independently, the same or different, H, a C₁-C₈ alkyl group (or a C₁-C₄ alkyl group in some embodiments) or a phenyl group, wherein u is an integer of 2 or 3, v is an integer of 0, 2 or 3 and x is an integer of 0, 2 or 3, and wherein at least one of Rₐ and R_{b} is not H, and R_{c} is a labile amino protecting group selected from the group of a carbobenzyloxy group, a p-methoxybenzyl carbonyl group, a tert-butyloxycarbonyl group, a 9-fluorenylmethyloxycarbonyl group, an acetyl group, a benzoyl group, a benzyl group, a carbamate group, a p-methoxybenzyl group, a 3,4-dimethoxybenzyl group, a p-methoxyphenyl group, a tosyl group, a mesyl group, a dimethoxytrityl group, a [bis-(4-methoxyphenyl)phenylmethyl] group, a pivaloyl group, a trimethylsilyl group, a tert-butyldimethylsilyl group, a tri-iso-propylsilyloxymethyl group, and a triisopropylsilyl group. In a number of embodiments, R_{c} is a trimethylsilyl group or a pivaloyl group. In a number of embodiments, R_{c} is a pivaloylmethyl group.

In another aspect, a method of synthesizing a compound having the formula:

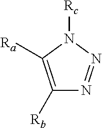

wherein Rₐ, and R_{b} are independently, the same or different, H, a halo group, a C₁-C₈ alkyl group, a C₂-C₈ alkenyl group, C₂-C₈ alkynyl group, —Si(R_{d}RₑR_{f}), —(R_{g})Si(R_{d}RₑR_{f}), wherein R_{g} is a C₂-C₆ alkylene group, and R_{d}, Rₑ and R_{f} are independently a C₁-C₈ alkyl group or a C₁-C₁₀ aryl group, —(Rₕ)ORᵢ wherein Rₕ is a C₂-C₆ alkylene group and Rᵢ is a C₁-C₈ alkyl group, a C₁-C₈ haloalkyl, a C₂-C₈ alkenyl group, a phenyl group or an C₂-C₈ alkynyl group, —ORᵢ, a cyano group, a C₁-C₆ cyanoalkyl group, a C₁-C₈ hydroxyalkyl group, (Rⱼ)(O)ORₖ, wherein Rⱼ is an C₂-C₆ alkylene group and Rₖ is a C₁-C₈ alkyl group, a C₁-C₈ haloalkyl group, a C₂-C₈ alkenyl group or a C₂-C₈ alkynyl group, an amine group, a C₁-C₈ alkylamine, a C₁-C₈ dialkylamine group, a C₁-C₈ haloalkyl group, a C₆-C₁₀ aryl group, a C₈-C₁₆ alkylaryl group, a C₈-C₁₆ arylalkyl group, —(R₁₅)ₙ[Si(Rₗ)(Rₘ)O]ₚSi(R_{q}RᵣRₛ), wherein n is an integer from 1 to 5, p is an integer from 1 to 9, wherein R₁₅ is a C₂-C₃ alkylene group, and Rₗ, Rₘ, R_{q}, Rᵣ and Rₛ are independently, the same or different, H, a C₁-C₈ alkyl group (or a C₁-C₄ alkyl group in some embodiments) or a phenyl group, or —R₁₆O[C(R₁₇)(R₁₈)]ᵤO[C(R₁₉)(R₂₀)]ᵥO[C(R₂₁)(R₂₂)]ₓOR₂₃, wherein R₁₆ is a C₂-C₆ alkylene group, R₁₇, R₁₈, R₁₉, R₂₀, R₂₁, R₂₂ and R₂₃ are independently, the same or different, H, a C₁-C₈ alkyl group (or a C₁-C₄ alkyl group in some embodiments) or a phenyl group, wherein u is an integer of 2 or 3, v is an integer of 0, 2 or 3 and x is an integer of 0, 2 or 3, and wherein at least one of Rₐ and R_{b} is not H, and R_{c} is H or a labile amino protecting group selected from the group of a carbobenzyloxy group, a p-methoxybenzyl carbonyl group, a tert-butyloxycarbonyl group, a 9-fluorenylmethyloxycarbonyl group, an acetyl group, a benzoyl group, a benzyl group, a carbamate group, a p-methoxybenzyl group, a 3,4-dimethoxybenzyl group, a p-methoxyphenyl group, a tosyl group, a mesyl group, a dimethoxytrityl group, a [bis-(4-methoxyphenyl)phenylmethyl] group, a pivaloyl group, a trimethylsilyl group, a tert-butyldimethylsilyl group, a tri-iso-propylsilyloxymethyl group, and a triisopropylsilyl group, wherein at least one of Rₐ and R_{b} is not H, includes:

reacting an alkyne having the formula:

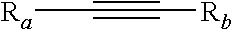

with an azide having the formula R_{c}—N₃ in a 3+2 cycloaddition reaction. The alkyne may, for example, be reacted with the azide in the presence of a catalyst or under heat. In a number of embodiments, the catalyst is a copper catalyst or a ruthenium catalyst. In a number of embodiments, R_{c} is a trimethylsilyl group or a pivaloyl group. In a number of embodiments, R_{c} is a pivaloylmethyl group. In the case that R_{c} is H, the method may further include first synthesizing the compound wherein R_{c} is the labile amino protecting group and subsequently removing the labile amino protecting group.

In a further aspect, a method of separating carbon dioxide from a gas mixture, includes placing an ionic liquid comprising a mono- or di-substituted 1,2,3-triazolide anion and an ionic liquid cation in fluid connection with the gas mixture. The ionic liquid may, for example, separate carbon dioxide from the gas mixture via sorption and the method may further include stripping carbon dioxide from the ionic liquid. Sorption of the carbon dioxide may, for example, include chemisorption. As described above, the ionic liquid may, for example, have the formula:

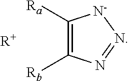

In still a further aspect, a method of carrying out a base-catalyzed reaction includes carrying out the reaction in an ionic liquid comprising a mono- or di-substituted 1,2,3-triazolide anion and an ionic liquid cation. As described above, the ionic liquid may, for example, have the formula:

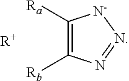

The terms “alkyl”, “aryl” and other groups refer generally to both unsubstituted and substituted groups unless specified to the contrary. Unless otherwise specified, alkyl groups are hydrocarbon groups and are, for example, C₁-C₁₀ (that is, having 1 to 10 carbon atoms) or C₁-C₈, and can be branched or unbranched, acyclic or cyclic. The above definition of an alkyl group and other definitions apply also when the group is a substituent on another group (for example, an alkyl group as a substituent of an alkylamino group or a dialkylamino group). The term “aryl” refers to C₆-C₁₀ aryl group (phenyl or naphthyl). As used herein, the terms “halogen” or “halo” refer to fluoro, chloro, bromo and iodo. In a number of embodiments, halo group are fluoro or chloro.

The term “alkoxy” refers to —ORᵢ, wherein Rᵢ is an alkyl group. The term “alkenyl” refers to a straight or branched chain hydrocarbon group with at least one double bond, with, for example, 2-10 carbon atoms or 2-8 carbon atoms (for example, —CH═CHR or —CH₂CH═CHR). The term “alkynyl” refers to a straight or branched chain hydrocarbon group with at least one triple bond, with, for example, 2-10 carbon atoms or 2-8 carbon atoms (for example, —C≡CR or CH₂—C≡CR). The terms “alkylene,” “alkenylene” and “alkynylene” refer to bivalent forms of alkyl, alkenyl and alkynyl groups, respectively, which may be straight or branched. The groups set forth above, may be substituted with a wide variety of substituents.

The present systems, methods and compositions along with the attributes and attendant advantages thereof, will best be appreciated and understood in view of the following detailed description taken in conjunction with the accompanying drawings.

### BRIEF DESCRIPTION OF THE DRAWINGS

FIG. 1A illustrates a reaction scheme for preparation of substituted-1,2,3,-triazolide ionic liquids hereof.

FIG. 1B illustrates a number of representative cations suitable for use in forming substituted-1,2,3,-triazolide ionic liquids hereof

FIG. 2 illustrates several representative pivaloylmethyl-4-substitutued-1,2,3,-triazoles synthesized.

FIG. 3 illustrates H-4-substituted-1,2,3,-triazoles synthesized from the compounds of FIG. 2.

FIG. 4A illustrates several representative tertbutyl phosphonium-4-substituted-1,2,3-triazolide ionic liquids synthesized from the compounds of FIG. 3.

FIG. 4B illustrates several representative tertbutyl phosphonium-4,5-substituted-1,2,3-triazolide ionic liquids synthesized in studies hereof.

FIG. 5 illustrates the solubility of CO₂ in ionic liquids 1c-5c of FIG. 4A and ionic liquids 7-9 of FIG. 4B at 30° C.

### DETAILED DESCRIPTION

It will be readily understood that the components of the embodiments, as generally described and illustrated in the figures herein, may be arranged and designed in a wide variety of different configurations in addition to the described example embodiments. Thus, the following more detailed description of the example embodiments, as represented in the figures, is not intended to limit the scope of the embodiments, as claimed, but is merely representative of example embodiments.

Reference throughout this specification to “one embodiment” or “an embodiment” (or the like) means that a particular feature, structure, or characteristic described in connection with the embodiment is included in at least one embodiment. Thus, the appearance of the phrases “in one embodiment” or “in an embodiment” or the like in various places throughout this specification are not necessarily all referring to the same embodiment.

Furthermore, described features, structures, or characteristics may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, et cetera. In other instances, well known structures, materials, or operations are not shown or described in detail to avoid obfuscation.

As used herein and in the appended claims, the singular forms “a,” “an”, and “the” include plural references unless the context clearly dictates otherwise. Thus, for example, reference to “an ionic liquid” includes a plurality of such ionic liquids and equivalents thereof known to those skilled in the art, and so forth, and reference to “the ionic liquid” is a reference to one or more such ionic liquids and equivalents thereof known to those skilled in the art, and so forth.

IL-based carbon capture system may, for example, reduce viscosity issues by reducing or eliminating the potential detrimental formation of salt bridges and hydrogen bonds. To avoid increasing the viscosity with high CO₂ capacity (via formation of salt-bridges between the anions) aprotic heterocyclic anions (AHAs) may be used to efficiently and reversibly capture CO₂. Aprotic heterocyclic anions may, for example, be tuned for stability, CO₂ absorption enthalpy, and CO₂ capacity.

Inspection of the pKₐs of azoles including 1,2,3-triazole, tetrazole and 1,2,4-triazole, shows that 1,2,3-triazole are of intermediate basicity (Table1). Table 1 sets forth the pKa values of various azoles in DMSO. Tetrazole does not successfully capture CO₂, whereas 1,2,4-triazole does.

TABLE 1

| | | | 1,2,4- | | |
|---|---|---|---|---|---|
| pyrrole | pyrazole | imidazole | triazole | 1,2,3-Triazole | tetrazole |
| 23.0 | 19.8 | 18.6 | 14.8 | 13.9 | 8.2 |

Substituted 1,2,3-triazoles can serve as AHA IL precursors and fortuitously possess a pKₐ amenable to the formation of an anion which can react reversibly with CO₂. Azoles which are too basic tend to interact too strongly with H₂O and bind CO₂ irreversibly. Azoles which are insufficiently basic do not interact with CO₂ strongly enough to bind it. The pKₐ of 1,2,3-triazole falls in an intermediate range (Table 1), where these two trends may be balanced.

Although representative examples of 1,2,3-triazolide based ionic liquids are discussed in the context of CO₂ capture herein, the 1,2,3-triazolide based ionic liquids hereof may be used in other systems and/or processes. For example, 1,2,3-triazolide based ionic liquids hereof may be used as solvents/catalysts in a number of base-catalysed reaction schemes. In that regard, the basic nature of the triazolide anion may make the 1,2,3-triazolide based ionic liquids hereof suitable for use in, for example, acyl transfer and ring-opening polymerizations of cyclic esters and metal-free ring-opening polymerization of lactones, lactides, caprolactones, etc. Moreover, 1,2,3-triazolide based ionic liquids hereof may be used in supported ionic liquid membranes. For example, 1,2,3-triazolide based ionic liquids hereof may be supported upon a porous support such as a porous polymeric support. Further, 1,2,3-triazolide based ionic liquids may be used as reactants in various chemical reactions.

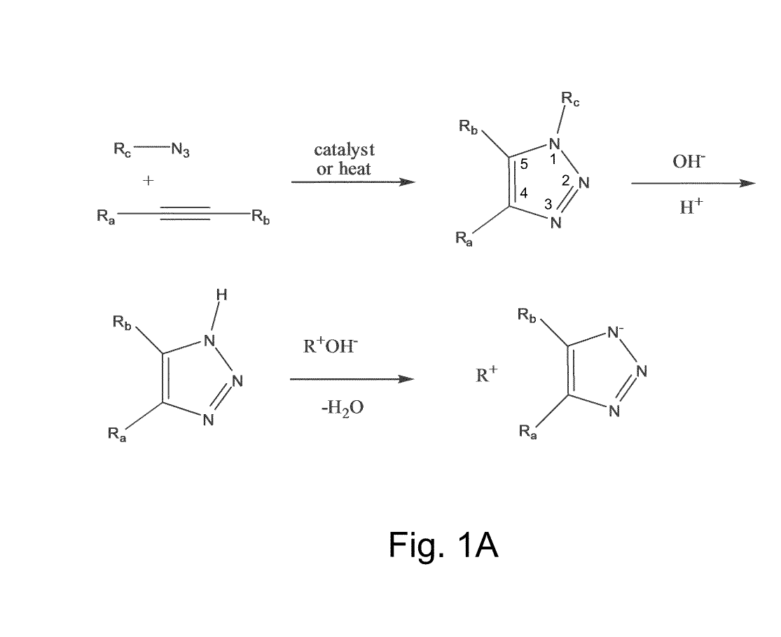

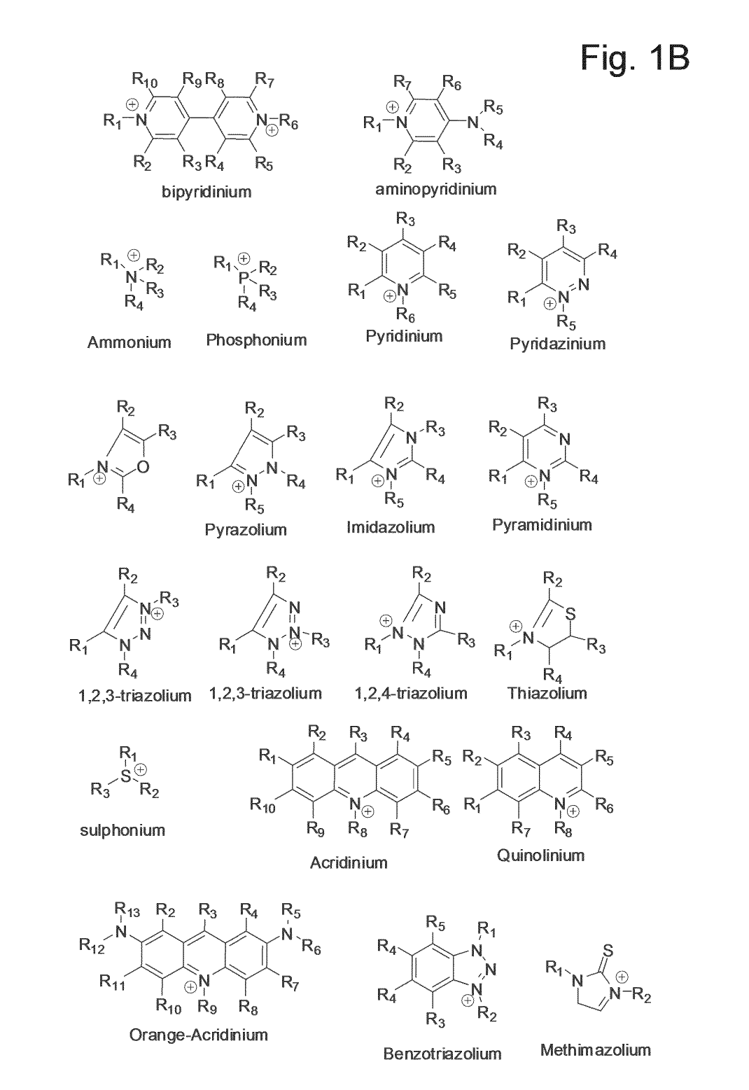

In a number of representative embodiments, we prepared triazolide based ionic liquids utilizing a [3+2] cycloaddition reaction which is capable of generating a wide variety of 1,2,3-triazoles with multiple functional groups located on the 1,2,3-triazole core. The general reaction scheme is illustrated in FIG. 1A. The cycloaddition reaction between azide and alkyne illustrated in FIG. 1A is highly tolerant of widely varying functional group and requires mild reaction conditions. In a number of embodiments, a Cu(I)-catalyzed Huisgen cycloaddition reaction was carried out in which Rb was H, yielding essentially pure 1,4-difunctional triazole regio-isomers. A ruthenium (Ru) catalyst may also be used. In a number of other embodiments, 1,4,5-trifunctional traizole compounds were formed via a thermal route without catalyst.

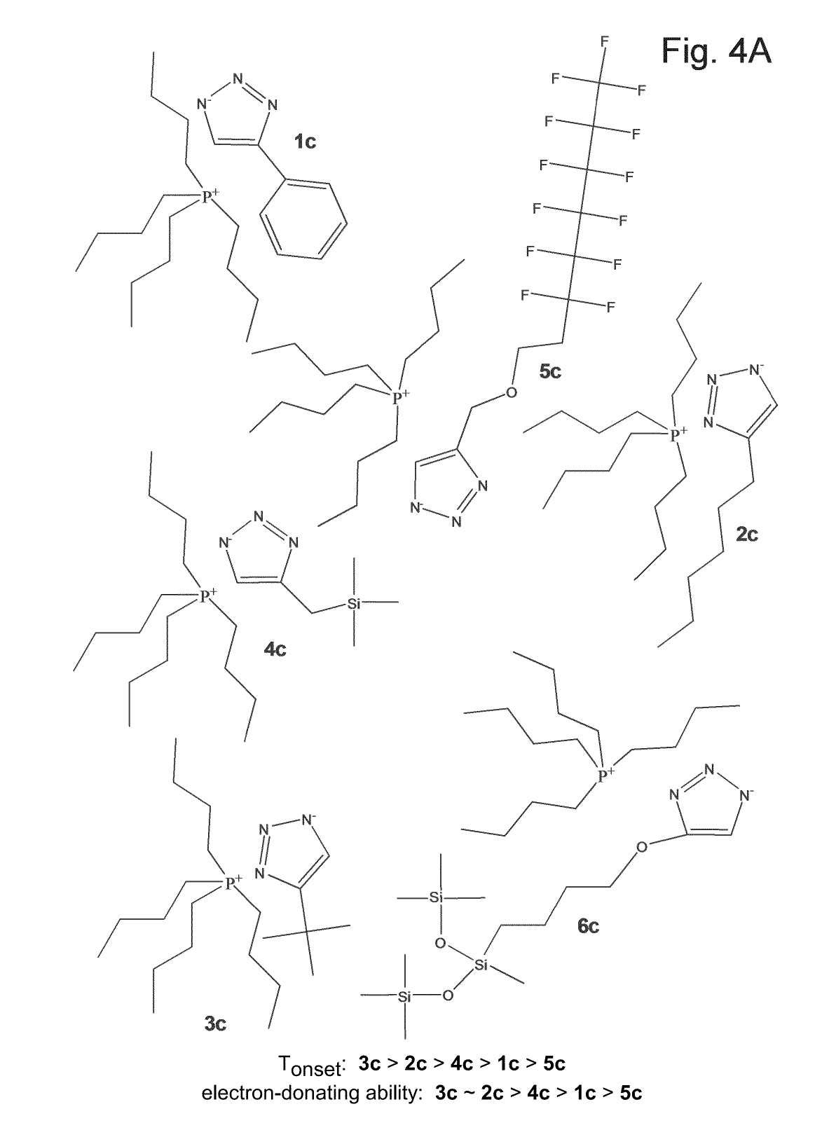

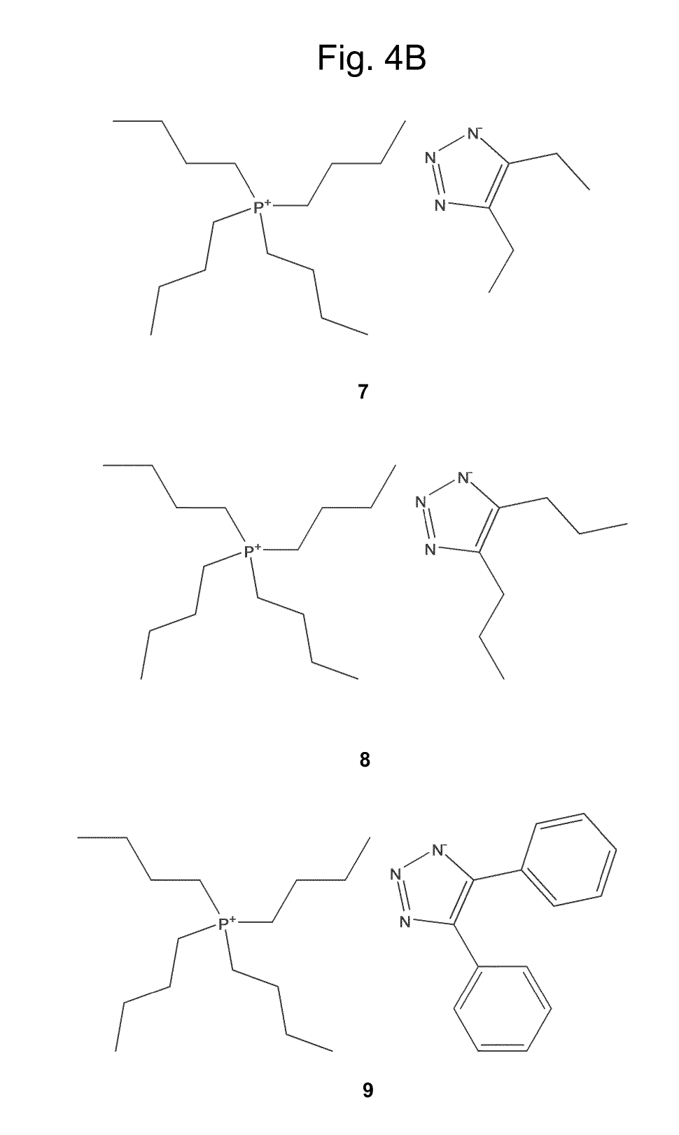

In the case that Rb in FIG. 4A is H, the Rₐ substitutent on the alkyne becomes the 4-substituent on 1,2,3-trazole. In a number of representative studies hereof, Rₐ was a phenyl group (Ph), a hexyl group (C₆H₁₃), a t-butyl group ((CH₃)₃C), a trimethylsilyl methyl group ((CH₃)₃SiCH₂), a bis(trimethylsiloxylbutoxymethyl group (((CH₃)₃SiO)₂Si(CH₃)(CH₂)₄OCH₂) and a tridecafluorooctyloxymethyl group (CH₃(CF₂)₅(CH₂)₂OCH₂). However, Rₐ and/or R_{b} may vary widely, including, for example, the substituents set forth above. The R_{c} substituent is, for example, a protecting group that can be removed to provide a protonated 4- and/or 5-substituted triazole. The resulting triazole is readily transformed into the protonated substituted triazole under mild conditions, which in turn is a facile precursor for a large family of ionic liquids. In a number of studied embodiments, the R_{c} group of the azide was an alkaline labile group such as a pivaloylmethyl (POM) group or a trimethylsilyl group (TMS).

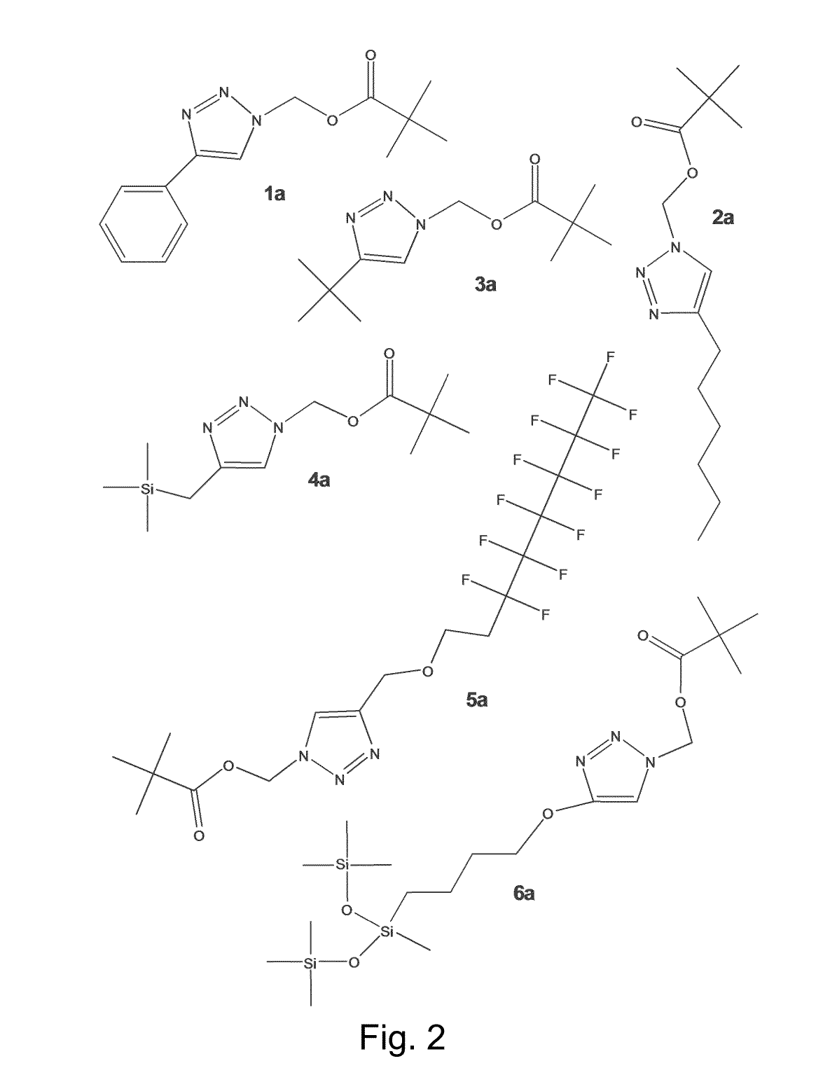

A number of triazolide-based AHA ionic liquids were prepared. As discussed above, the 1,4-disubstituted precursors structures prepared are shown in FIG. 1A using a Cu(I) catalyst. These molecules were converted to 1H-4-substituted-1,2,3-triazoles, as shown in FIG. 2, after alkaline hydrolysis in methanol-water mixture and were converted into ionic liquids by treatment with a hydroxide of an IL cation (tetrabutyl phosphonium hydroxide in a number of representative embodiments). The 1H-4-substituted 1,2,3-triazole and hydroxide undergo an acid-base reaction to form the ionic liquid product. Representative 4-substituted 1,2,3-triazolide AHA ionic liquids prepared in several studies hereof are shown in FIG. 4A. The ionic liquid formation may be extended to any other IL cation by using the appropriate hydroxide. A number of representative ionic liquid cations are illustrated in FIG. 1B and include, for example, a pyridinium cation, a bipyridinium cation, an amino pyridinium cation, a pyridazinium cation, an ozaxolium cation, a pyrazolium cation, an imidazolium cation, a pyramidinium cation, a triazolium cation, a thiazolium cation, an acridinium cation, a quinolinium cation, an isoquinolinium cation, an orange-acridinium cation, a benzotriazolium cation, a methimzolium cation, a sulfonium cation, an ammonium cation, or a phosphonium cation. Such cations may, for example, be widely substituted. In a number of embodiments, R₁ through R₁₃ of FIG. 1B are independently (the same or different) a C₁-C₆ alkyl group, a C₁-C₆ alkenyl group, hydroxyalkyl group, a haloalkyl group, an alkoxylalkyl; a C₆-C₁₀ aryl group or a C₈-C₁₆ alkylenearyl group. By, for example, varying the electronic nature of the substituents at the 4-position and/or 5-position on the triazole ring, stabilization of the negative charge on the resulting anion can be affected and increased molar volume can be achieved, which may, for example, be important for solvent applications.

1H-4,5 disubstituted 1,2,3-triazoles were prepared by the thermal [3+2] cycloaddition of azide R_{c}N₃ with internal alkynes RₐC≡CR_{b}, in which the azide and alkyne (2:1 molar ratio) were mixed in a sealed Schlenk tube under N₂, then heated to 180-200 C for 5-7 days. In a number of studied embodiments R_{c} was a trimethylsilyl group. The reaction product was stirred in water at 50° C. to hydrolyze the trimethylsilyl group. The 1H-4,5 disubstituted 1,2,3-triazoles were converted into ionic liquids by performing a dehydration reaction between the triazoles and an ionic liquid cation hydroxide (for example, tetrabutylphosphonium hydroxide) as discussed above. Representative 4,5-substituted 1,2,3-triazolide AHA ionic liquids prepared in several studies hereof are shown in FIG. 4B. In the representative embodiments of FIG. 4B, the 1,2,3, triazolide is symmetrically substituted (that is, the 4- and 5-substituents are the same). However, 4,5-substituted 1,2,3-triazolide AHA ionic liquids hereof may be asymmetrically substituted.

A set of five AHA ILs based on 4-substituted-1,2,3-triazole was extensively characterized. The representative triazoles prepared were chosen to include a variety of substituents, with both electron-withdrawing (phenyl—1b) and electron-donating groups at the 4-position (n-hexyl and t-butyl—2b, 3b). A silyl- and fluorinated ether-substituted triazole (4b, 5b) were also included to observe the effect of hydrophobic substituents on the AHA IL.

The physical properties of the 4-substituted triazolide AHA ionic liquids were measured, with the results listed in Table 2. The densities (ρ) of all of the ionic liquids, except for the fluorinated 5c, were approximately 1 g/mL; the molar volumes (Vm) listed were calculated from these density values. Karl Fisher (KF) titration of the ILs revealed that after vacuum drying to constant weight at 50° C., 0.9 to 2.4 wt % water still remained in the ILs.

TABLE 2

| | | | | | wt % | | mol ab- |
|---|---|---|---|---|---|---|---|
| | | | Vₘ, | | H₂O | mol % | sorbed |
| | FW, | ρ,^{a} | mL/ | Tₒₙₛₑₜ, | (by | H₂O | CO₂/ |
| IL | g/mol | g/mL | mol | ° C. | KF) | (by KF) | mol IL |
| 1c | 403.58 | 0.9836 | 402.7 | 238 | 1.06 | 19.4 | 0.10 |
| 2c | 411.65 | 0.9324 | 431.2 | 185 | 2.35 | 35.5 | 0.40 |
| 3c | 383.59 | 0.9290 | 401.2 | 151 | 0.914 | 16.4 | 0.07 |
| 4c | 413.70 | 0.9400 | 416.6 | 227 | 1.04 | 19.5 | 0.30 |
| 5c | 704.61 | 1.2141 | 578.6 | 263 | 0.973 | 27.8 | 0.23 |

Thermal analysis of the decomposition of the ILs 1c-5c showed that electron-donating groups (1c, 5c) on the triazole ring begin to decompose (Tₒₙₛₑₜ) at lower temperatures than those with electron-withdrawing groups (2c-4c). Alkyl groups on 2c and 3c cause the ILs to begin thermal decomposition at a substantially cooler temperature than for 1c and 5c. A comparison of Tₒₙₛₑₜ and substituent electron-donating ability for ILs 1c-5c is shown at the bottom of FIG. 4A. The onset of thermal decomposition for ILs 1c-5c were found to be comparable with results reported for [P₆₆₆₁₄][imidazolide] and [P₆₆₆₁₄][pyrazolide] (T_{dec}=252 and 182° C., respectively).

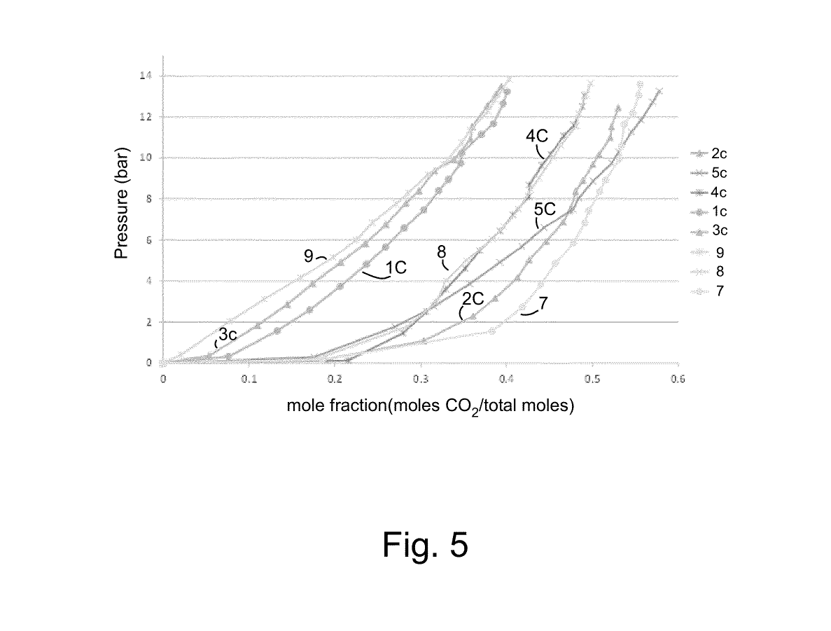

The results of CO₂ solubility experiments for ionic liquids 1c-5c are summarized in FIG. 5. Without limitation to any mechanism, the amounts of CO₂ absorbed by ionic liquids 1c-5c are consistent with what would be expected for the steric bulk of the substituent located on the azole ring. In the cases of 2c and 5c, the linear geometry of the hexyl- and fluoroether-groups exert less steric crowding adjacent to the azole ring, leaving the incoming CO₂ a greater amount of free space in which to react. In the case of 3c, the branched nature of the t-butyl group maximizes the steric crowding near the azole ring and prevents the CO₂ from having an easy approach during reaction. That 4c (TMS—electron donating group) has a greater CO₂ capacity than 1c (Ph—electron withdrawing group) suggests that while steric crowding is an important factor in determining CO₂ capacity, electronic effects also exert an effect on the CO₂ reaction.

One method for quantifying the relative steric crowding near the triazolide ring is the angular spread of the phosphine cone angle in comparable trialkyl phosphines. The cone angle is smaller for straight chain substituents (132° for 2c and 5c) and grows larger for highly branched substituents (180°-182° for 3c and 4c). The cone angle for phenyl rings lies approximately half way between the linear and branched alkyl groups (145° for 1c).

The ¹³C NMR spectra of neat 1,2,3-triazolide ILs were all collected before and after exposure to ¹³CO₂. Extra ¹³C peaks were observed in the neat NMR spectra of 3c before and after ¹³CO₂ exposure, although they were not seen in the solution spectra. These peaks may arise from the formation of anionic dimers.

Samples of ILs 1c-5c were deposited as neat films on CaF₂ and were observed by transmission FTIR under varying pressures of CO₂. ILs 1c-4c were first evacuated (<10⁻⁶ Torr), then dosed with 10 and 100 Torr CO₂ and new features were observed to appear in 1c-4c in the carbamate regions of the spectrum (1100-1200, 1600-1800 cm⁻¹). It was not possible to prepare sufficiently thin layer of 5c to obtain useful FTIR spectra. New features were seen to appear in the spectra after 10 Torr CO₂ at 1754, 1628, and 1280 cm⁻¹. These features disappeared when the sample was again evacuated to 10⁻⁶ Torr, suggesting that the CO₂ adsorption is reversible for triazolide ILs as well as other azolide ILs. Similar results were obtained for 1c, 3c, and 4c.

The mass spectra of 4-substituted 1,2,3-triazolide IL solutions in methanol were all obtained and the results are listed in Table 3. In Table 3, R⁺=IL cation, A⁻=IL anion, and nd=not detected. In all cases, the molecular ion for the cations were observed in positive mode, and the molecular ion for the triazolide anions were observed in negative mode, confirming the identities of all products. In addition to the molecular ions, evidence of dimeric anion species was observed for all 4-substituted 1,2,3-triazolide ILs except 4c, which yielded a molecular ion signal with difficulty. The reactive ring proton at C₅ is the likely site for any side reactions which may be occurring.

TABLE 3

| sample | | R⁺ | A⁻ | (2A + H)⁻ | (2A + C)⁻ |
|---|---|---|---|---|---|
| 1c | calc'd | 259.43 | 144.15 | 289.31 | 547.73 |
| | found | 259.26 | 144.07 | Nd | 547.37 |
| 2c | calc'd | 259.43 | 152.22 | 305.45 | 563.87 |
| | found | 259.26 | 152.12 | 305.24 | 563.49 |
| 3c | calc'd | 259.43 | 124.16 | 249.33 | 507.75 |
| | found | 259.26 | 124.09 | 249.20 | 507.43 |
| 4c | calc'd | 259.43 | 154.27 | 309.55 | 567.97 |
| | found | 259.26 | 154.10 | Nd | nd |
| 5c | calc'd | 259.43 | 444.17 | 889.35 | 1147.77 |

To further study the relative effect of electron donating and withdrawing substitution of the 1,2,3-triazolide system on CO₂ interaction, the 4,5-substituted 1,2,3-triazolide AHA ionic liquids were synthesized and characterized for CO₂ solubility. FIG. 5 illustrates the solubility of CO₂ in ionic liquids 1c-5c of FIG. 4A and ionic liquids 7-9 of FIG. 4B at 30° C. In the studies of FIG. 5, dosing with a known amount of CO₂ was repeated to obtain an isotherm over a range of pressure. As seen from FIG. 5, disubstitution with electron withdrawing phenyl groups resulted in the lowest levels chemisorption of CO₂, while disubstitution with electron donating (and relatively small) ethyl groups resulted in the highest levels of chemisorption of CO₂.

Furthermore, the potential for dimeric anion formation is reduced or eliminated in the case of 4,5-substituted (disubstituted) 1,2,3-triazolide AHA ionic liquids. Without limitation to any mechanism, elimination of the acidic proton present in 4-substituted (monosubstituted) 1,2,3-triazolide AHA ionic liquids may prevent the formation of dimers. Disubstituted 1,2,3-triazolide AHA ionic liquids also exhibit lower viscosities than monosubstituted 1,2,3-triazolide AHA ionic liquids of the same molecular weight, which may, for example, be a result of the absence of a hydrogen bonding proton. Moreover, the lack of such a proton may result in greater stability in the case of disubstituted 1,2,3-triazolide AHA ionic liquids.

Although the 1,2,3-triazolide AHA ionic liquids hereof interact with water, unlike previously described AHA ionic liquids, the 1,2,3-triazolide AHA ionic liquids are water insoluble with a broad range of cations. All 1,2,3-triazolide AHA ionic liquids synthesized were water insoluble. Water uptake and interaction is lower in the case of 4,5-substituted 1,2,3-triazolide AHA ionic liquids than for 4-substituted 1,2,3-triazolide AHA ionic liquids.

The aprotic ionic liquids based on substituted 1,2,3-triazoles hereof absorb CO₂ at ambient conditions. By selection of the substituent at the 4-position and/or 5-position, the basicity of and the reactivity of the resulting 1,2,3-triazolide anions toward, for example, CO₂ can be affected/adjusted. For example, depending upon the electronic nature and steric bulk of the substituent(s), CO₂ chemisorption levels may be adjusted. In a number of embodiments, 4-position and/or 5-position substituent(s) may be chosen to be relatively small (minimizing steric bulk) and electron donating (increasing activity) to maximize CO₂ chemisorption. Electron withdrawing groups may, however, be used to improve thermal and/or chemical stability in some uses.

### Experimental

General Information.

The ¹H, ¹³C, ¹⁹F, and ³¹P NMR spectroscopic analyses were completed using Bruker Avance III 300 or 400 MHz spectrometer. Fourier transform infrared (FTIR) spectra were collected on a Nicolet Spectrum 100 with an attenuated total reflectance (ATR) apparatus. Density measurements of the ionic liquids were performed using a Micromeritics Accupyc II 1340 pycnometer. Water content was determined using a Metrohm 860 Karl Fisher (KF) Thermoprep titration unit, equipped with an 831 KF Coulometer; ionic liquid samples which had undergone KF titration were treated as water-free samples. Viscosity measurements were made at ambient temperatures using a Rheosense Inc. μVisc unit.

Thermogravimetric Analysis.

Thermal decomposition studies were performed on a Mettler Toledo TGA/DSC 1 STARe System, equipped with a GC 200 gas controller. Thermal decompositions were conducted under N2 purge from 22° C. to 500° C. at a ramp rate of 10° C. per minute. The onset of thermal decomposition (Tₒₙₛₑₜ) was evaluated as the temperature at which the signal baseline began to drop to a value exceeding that observed in the preceding baseline noise.

Mass Spectrometry Analysis.

Mass spectrometry (MS) analysis was performed using an Agilent 1290 LC system and 6520 Q-TOF (quadrupole time of flight) detector (Agilent Technology). A 95% MeOH/5% H₂O mixture was used as the mobile phase. A 0.1 μL sample was injected using an autosampler and introduced into the chromatograph using direct infusion (no column) with a mobile phase flow rate of 0.4 mL/min. An ESI (electrospray ionization) source was used to introduce the samples into the Q-TOF with the following settings: fragmentor voltage=175 V, skimmer voltage=65 V, drying gas flow rate and temperatures of 12 L/min and 300° C., respectively. The acquisition range was 100-3000 m/z with a scanning rate of 1.03 spectra/s. Each sample was run for 4 min. in both positive and negative polarities. MeOH blanks were run between injections to decrease the amount of carryover that may occur. The chromatograms were integrated and extracted for MS spectra to determine the masses of the cations and anions.

FTIR Spectroscopy.

Variable pressure FTIR experiments performed were conducted on a Nicolet Nexus 6700 ESP spectrometer (Thermo Fisher Scientific, Waltham, Mass.), equipped with a wide-band mercury cadmium telluride (MCT) detector cooled by liquid N₂. Low pressure infrared studies were performed in a stainless steel vacuum chamber equipped with a turbo-molecular pump, roughing pump, precision leak valve, and differentially pumped optical windows (KBr). System pressure was measured using an ionization gauge. The system base pressure was in the 10⁻⁷ Torr range after an overnight evacuation. The sample was manipulated inside the chamber with an XYZ translation stage and a rotation stage. Samples were prepared on CaF₂ windows using a Janis Research (Wilmington, Mass.) copper sample holder, accommodating one blank and one sample window.

CO₂ Absorption.

Solubility measurements were performed using a PCT-Pro 2000 apparatus from Setaram Inc. The PCT-Pro 2000 is a Sievert's apparatus, which determines gas solubility in a sample by charging a sealed sample chamber of known volume with a known quantity of CO₂. The CO₂-charged sample chamber is isolated from the rest of the system and the pressure drop in the chamber is measured. The drop in pressure due to CO₂ absorption into the liquid is then measured, and the quantity of CO₂ absorbed into the liquid is determined from an equation of state, in this case, the NIST Standard Reference Database 23.

For all tests, between 0.2 g and 0.4 g of sample was loaded into the sample chamber. The sample was held under a dynamic vacuum for 4 hours prior to starting a test. During this evacuation, the sample was heated to 30° C. and was stirred at 300 rpm. After evacuation, testing was initiated by dosing the sample chamber with a known amount of CO₂, and the sample was allowed to equilibrate for at least 4 hours. Dosing was repeated to obtain an isotherm over a pressure range of 0 to 10 bar. Throughout all tests, the sample was maintained at 30° C. and was stirred at 300 rpm.

Materials.

Unless otherwise stated, ACS reagent grade chemicals and solvents were obtained from Aldrich and used without further purification. Tetrabutyl phosphonium hydroxide (40% aqueous solution) was obtained from TCI America and was used as received.

### General method for the synthesis of 1-POM-4-phenyl-1,2,3-triazole (1a)

Azido methylpivalate was synthesized from sodium azide and chloromethyl pivalate in water. Azido methylpivalate (5.00 g, 31.8 mmol), phenyl acetylene (4.12 g, 40.4 mmol), triethyl amine (3.63 g, 35.9 mmol), and 3% Cu/charcoal (3.05 g, 1.44 mmol) were placed in a Schlenk tube, dissolved in 15 mL dioxane, and heated overnight at 80° C. This mixture was filtered to remove the catalyst, and then evaporated and vacuum dried to give a brown solid. The residue was dissolved in minimal Et₂O, filtered through a 0.2 μm syringe filter, evaporated, and vacuum dried to give 1a as a white solid (7.25 g, 28.0 mmol, 88% yield). ¹H NMR (300 MHz, CDCl₃): δ 8.04 (s, 1H, triazole C—H), 7.87 (m, 2H, o-Ph), 7.45 (m, 2H, m-Ph), 7.42 (m, 1H, p-Ph), 6.29 (s, 2H, piv-CH2), 1.21 (s, 9H, piv-CH₃); ¹³C NMR (75.5 MHz, CDCl3): δ 178.0 (C═O), 148.3 (triazole), 130.0 (triazole), 128.9, 128.5, 125.8, 120.9, 69.7, 38.8, 26.8; FTIR (ATR film): 1737 cm⁻¹ (C═O); Calc'd for C₁₄H₁₈N₃O₂⁺ ([M+H]⁺): 260, Found: 260.

### General method for the synthesis of 1H-4-phenyl-1,2,3-triazole (1b)

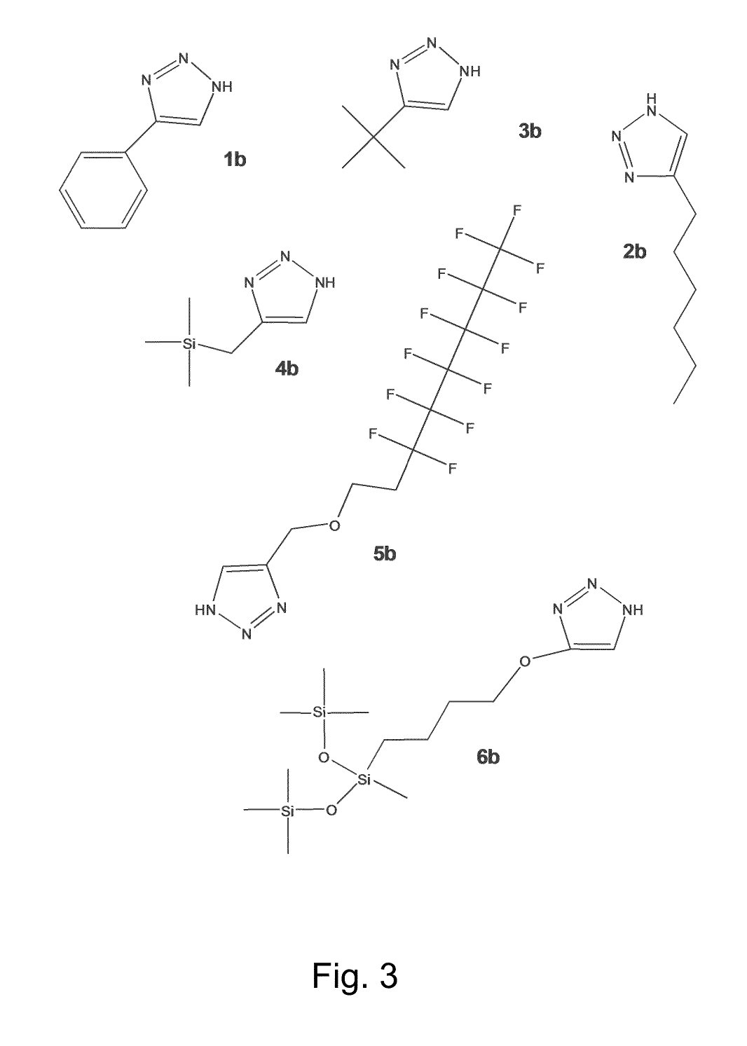

The structures for a number of molecules synthesized by this method are shown in FIG. 3. KOH (7.46 g, 133 mmol) and 1a (7.25 g, 28.0 mmol) were stirred in 50 mL 1:1 MeOH:water in air for 2 hr at room temperature, then was neutralized with 1 M HCl (100 mL, 100 mmol) to form a cloudy white solution. The mixture was filtered and the solids were rinsed with 300 mL water and vacuum dried to give off-white solids (3.48 g, 24.1 mmol, 86% yield). ¹H NMR (400 MHz, CDCl₃): δ 8.03 (s, 1H, triazole C—H), 7.83 (m, 2H, o-Ph), 7.47 (m, 2H, m-Ph), 7.40 (m, 1H, p-Ph); ¹³C NMR (101 MHz, CDCl₃): δ 148.9 (triazole), 132.2 (triazole), 129.0, 128.9, 126.2, 126.1; FTIR (ATR film): 3159, 3114, 2845, 1466, 1453, 1131, 1082, 1003, 972, 915, 873, 764, 693, 517 cm−1; ESI MS+/− (MeOH, m/z) Calc'd for C₈H₈N₃⁺ ([M+H]⁺): 146.17, Found: 145.95.

### General method for the synthesis of tetrabutyl phosphonium 4-phenyl-1,2,3-triazolide ([P₄₄₄₄][4-Ph-1,2,3-TZ]) (1c)

The structures for a number of 4-substituted triazolide ionic liquids synthesized by this method are shown in FIG. 4A. A solution of 1b (1.63 g, 11.2 mmol) in 25 mL EtOH was treated with 40% tetrabutyl phosphonium hydroxide ([P₄₄₄₄]OH) in water (7.84 g, 11.3 mmol) and was stirred at 50° C. for 6 hr. The solvent was then evaporated and the residue was vacuum dried at 50° C. until constant weight. The product was taken up in 10 mL EtOAc, filtered through a 0.2 μm syringe filter, evaporated, and vacuum dried to give a brown liquid (4.81 g, 106% yield). This liquid was then purified by refluxing over activated charcoal in MeOH at 65° C. for 24 hr. Evaporation and vacuum drying at 50° C. gave a pale red-brown liquid. ¹H NMR (700 MHz, DMSO): δ 7.69 (m, 2H, o-Ph), 7.60 (s, 1H, triazole C—H), 7.26 (m, 2H, m-Ph), 7.04 (m, 1H, p-Ph), 2.16 (m, 8H, P—CH₂), 1.41 (m, 16H, P—CH₂), 0.90 (t, 12H, P—CH₃); ¹³C NMR (101 MHz, DMSO): δ 142.8 (triazole), 135.7 (triazole), 128.1, 126.9, 124.4, 124.3, 23.2 (dd, P—CH2), 17.3 (d, 48.5 Hz, P—CH₂), 13.2 (P—CH₃); ³¹P NMR (162 MHz, DMSO): δ 33.6; FTIR (ATR film): 2958, 2928, 2871, 1603, 1465, 1379, 1096, 1047, 965, 906, 763, 718, 696, 682, 605, 512 cm⁻¹; ESI MS+/− (MeOH, m/z) Calc'd for C₂₂H₄₃N₃P: 403.58; Calc'd for cation C₁₆H₃₆P⁺ (M⁺): 259.43, Found: 259.27; Calc'd for anion C₈H₆N₃⁻ (M⁻): 144.15, Found: 144.07.

### General method for the synthesis of 1H-4,5-Disubstituted-1,2,3-Triazole

The thermal cycloaddition of trimethylsilyl azide (Me₃Si—N₃) with symmetrical internal alkynes R^{a}C≡CR_{b}, where Rₐ and R_{b}=ethyl (Et), propyl (Pr), and phenyl (Ph) was conducted using a method based on that cited by Birkofer and Wegner, Chem. Ber., 99, 2512 (1966), the disclosure of which is incorporated herein by reference, in which the azide and alkyne (2:1 molar ratio) are mixed in a sealed Schlenk tube under N₂, then heated to 180-200° C. for 5-7 days. After reaction, during which the solution passes from colorless to golden-brown, the solution is evaporated at 80° C. to remove unreacted azide and alkyne, then stirred in 50 mL water at 50° C. to hydrolyze the trimethylsilyl group. The hydrolyzed product is extracted into 3×40 mL hexane aliquots, washed with 100 mL water, dried over MgSO₄, filtered and vacuum dried to give yellow-brown waxy solids. Product is then dissolved in 15 mL DCM, filtered again via 0.2 um syringe filter, evaporated and vacuum dried to give clear, yellow-brown liquids (5-15% yield). In the case of Rₐ and R_{b}=phenyl (Ph), the product is purified by column chromatography (Biotage, EtOAc:hexane) to remove the unreacted PhC≡CPh.

### Tetrabutylphosphonium 4,5-Disubstituted-1,2,3-Triazolide Ionic Liquids

Disubstituted triazoles are converted into, for example, tetrabutylphosphonium ionic liquids by performing a dehydration reaction between the triazoles and tetrabutylphosphonium hydroxide (40% in water, 1.05 mole equiv) in minimal methanol (10 mL). The mixture is stirred in air at RT for 24 hr, then evaporated and dried to give a dark brown liquid. The product is taken up in 15 mL EtOAc, filtered via 0.2 um syringe filter, dried over MgSO₄, filtered again and vacuum dried to give clear viscous brown liquids (65-85% yield based on triazole). The liquids are dried on a Kugelrohr at 50° C. for 2-4 days to remove solvated water. In the case of R═Ph, the product becomes a waxy brown solid after all solvent and water has been removed.

The foregoing description and accompanying drawings set forth a number of representative embodiments at the present time. Various modifications, additions and alternative designs will, of course, become apparent to those skilled in the art in light of the foregoing teachings without departing from the scope hereof, which is indicated by the following claims rather than by the foregoing description. All changes and variations that fall within the meaning and range of equivalency of the claims are to be embraced within their scope.

What is claimed is:

1. A method of synthesizing an ionic liquid, comprising: reacting a 1,2,3-triazole comprising at least one of a 4-substituent or a 5-substituent with a hydroxide compound having the formula R⁺OH⁻ in a dehydration reaction, wherein R⁺ is an ionic liquid cation.

2. The method of claim 1 wherein R⁺ is a five-membered heterocyclic cation, an aromatic cation, a sulfonium cation, an ammonium cation, or a phosphonium cation.

3. The method of claim 1 wherein R⁺ is a pyridinium cation, a bipyridinium cation, an amino pyridinium cation, a pyridazinium cation, an ozaxolium cation, a pyrazolium cation, an imidazolium cation, a pyramidinium cation, a triazolium cation, a thiazolium cation, an acridinium cation, a quinolinium cation, an isoquinolinium cation, an orange-acridinium cation, a benzotriazolium cation, a methimzolium cation, a sulfonium cation, an ammonium cation, or a phosphonium cation.

4. The method of claim 1 wherein the R⁺ is a cation having the formula: 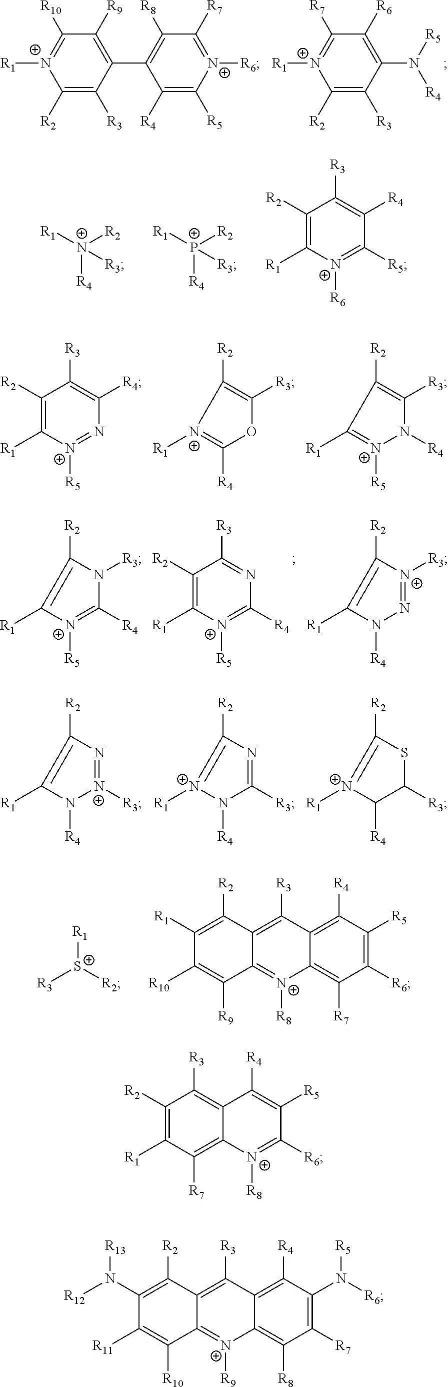 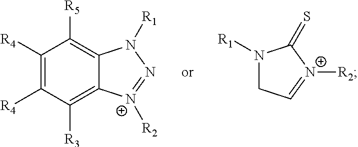
wherein R¹-R¹³ are independently, the same or different, a C₁-C₆ alkyl group, a C₁-C₆ alkenyl group, C₁-C₆ hydroxyalkyl group, a C₁-C₆ haloalkyl group, a C₂-C₈ alkoxylalkyl group, a C₆-C₁₀ aryl group a C₈-C₁₆ arylalkyl group or a C₈-C₁₆ alkylaryl group.

5. The method of claim 1 wherein R⁺ is an imidazolium cation, an ammonium cation, or a phosphonium cation.

6. The method of claim 1 wherein the R⁺ is a phosphonium cation.

7. The method of claim 1 wherein the 1,2,3-triazole comprising at least one of a 4-substituent or a 5-substituent has the formula: 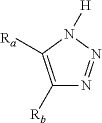
wherein Rₐ and R_{b} are independently H, an electron withdrawing group, an electron donating group, or a hydrophobic group and at least one of Rₐ, and R^{b} is not H.

8. The method of claim 7 wherein Rₐ, and R_{b} are independently, the same or different, H, a halo group, a C₁-C₈ alkyl group, a C₂-C₈ alkenyl group, C₂-C₈ alkynyl group, —Si(R_{d}RₑR_{f}), —(R_{g})Si(R_{d}RₑR_{f}), wherein R_{g} is a C₂-C₆ alkylene group, and R_{d}, Rₑ and R_{f} are independently a C₁-C₈ alkyl group or a C₁-C₁₀ aryl group, —(Rₕ)ORᵢ wherein Rₕ is a C₂-C₆ alkylene group and Rᵢ is a C₁-C₈ alkyl group, a C₁-C₈ haloalkyl, a C₂-C₈ alkenyl group, a phenyl group or an C₂-C₈ alkynyl group, —ORᵢ, a cyano group, a C₁-C₆ cyanoalkyl group, a C₁-C₈ hydroxyalkyl group, —(Rⱼ)(O)ORₖ, wherein Rⱼ is an C₂-C₆ alkylene group and Rₖ is a C₁-C₈ alkyl group, a C₁-C₈ haloalkyl group, a C₂-C₈ alkenyl group or a C₂-C₈ alkynyl group, an amine group, a C₁-C₈ alkylamine, a C₁-C₈ dialkylamine group, a C₁-C₈ haloalkyl group, a C₆-C₁₀ aryl group, a C₈-C₁₆ alkylaryl group, a C₈-C₁₆ arylalkyl group, —(R₁₅)ₙ[Si(Rₗ)(Rₘ)O]ₚSi(R_{q}RᵣRₛ), wherein n is an integer from 1 to 5, p is an integer from 1 to 9, wherein R₁₅ is a C₂-C₃ alkylene group, and Rₗ, Rₘ, R_{q}, Rᵣ and Rₛ are independently, the same or different, H, a C₁-C₈ alkyl group or a phenyl group, or —R₁₆O[C(R₁₇)(R₁₈)]ᵤO[C(R₁₉)(R₂₀)]ᵥO[C(R₂₁)(R₂₂)]ₓOR₂₃, wherein R₁₆ is a C₂-C₆ alkylene group, R₁₇, R₁₈, R₁₉, R₂₀ R₂₁, R₂₂ and R₂₃ are independently, the same or different, H, a C₁-C₈ alkyl group or a phenyl group, wherein u is an integer of 2 or 3, v is an integer of 0, 2 or 3 and x is an integer of 0, 2 or 3, and wherein at least one of Rₐ and R_{b} is not H.

9. The method of claim 8 wherein neither Rₐ, nor R_{b} is H.

10. The method of claim 8 wherein Rₐ, and R_{b} are independently H, Cl, F, a C₁-C₆ alkyl group, —CF₃, —COOCH₃, —CH₂OH, —CH₂OCH₃, —C≡N, —CH₂C≡N, —C≡CH, —CH₂C≡CH, —CHC═CH₂, a phenyl group.

11. The method of claim 10 wherein neither Rₐ, nor R_{b} is H.

12. The method of claim 8 wherein Rₐ, and R_{b} are independently H or a C₁-C₃ alkyl group.

13. The method of claim 8 wherein the 1,2,3-triazole comprising at least one of a 4-substituent or a 5-substituent is synthesized by reacting an alkyne having the formula: 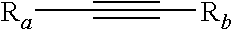
with an azide having the formula R_{c}—N₃ in a 3+2 cycloaddition reaction, wherein R_{c} is a labile amino protecting group selected from the group of a carbobenzyloxy group, a p-methoxybenzyl carbonyl group, a tert-butyloxycarbonyl group, a 9-fluorenylmethyloxycarbonyl group, an acetyl group, a benzoyl group, a benzyl group, a carbamate group, a p-methoxybenzyl group, a 3,4-dimethoxybenzyl group, a p-methoxyphenyl group, a tosyl group, a mesyl group, a dimethoxytrityl group, a [bis-(4-methoxyphenyl)phenylmethyl] group, a pivaloyl group, a trimethylsilyl group, a tert-butyldimethylsilyl group, a tri-iso-propylsilyloxymethyl group, and a triisopropylsilyl group, and subsequently removing the labile amino protecting group.

14. An ionic liquid comprising a mono- or di-substituted 1,2,3-triazole and an ionic liquid cation.

15. The ionic liquid of claim 12 having the formula: 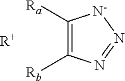
wherein R⁺ is s a five-membered heterocyclic cation, an aromatic cation, a sulfonium cation, an ammonium cation, or a phosphonium cation and Rₐ and R_{b} are independently H, an electron withdrawing group, an electron donating group, or a hydrophobic group, wherein at least one of Rₐ and R_{b} is not H.

16. The ionic liquid of claim 15 wherein R⁺ is a pyridinium cation, a bipyridinium cation, an amino pyridinium cation, a pyridazinium cation, an ozaxolium cation, a pyrazolium cation, an imidazolium cation, a pyramidinium cation, a triazolium cation, a thiazolium cation, an acridinium cation, a quinolinium cation, an isoquinolinium cation, an orange-acridinium cation, a benzotriazolium cation, a methimzolium cation, a sulfonium cation, an ammonium cation, or a phosphonium cation.

17. The ionic liquid of claim 15 wherein the R⁺ is a cation having the formula: 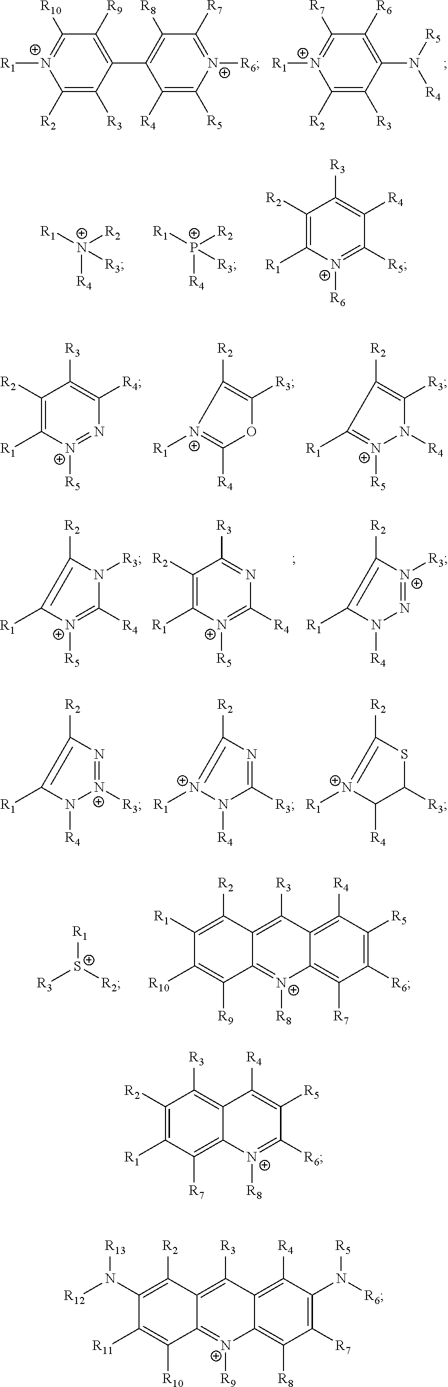 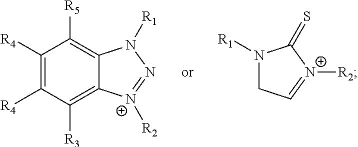
wherein R¹-R¹³ are independently selected from the group consisting of C₁-C₆ alkyl, alkenyl, hydroxyalkyl, haloalkyl, alkoxylalkyl; C₆-C₁₀ aryl or C₈-C₁₆ alkylenearyl; and mixtures thereof.

18. The ionic liquid of claim 15 wherein the R⁺ is an imidazolium cation, an ammonium cation, or a phosphonium cation.

19. The ionic liquid of claim 15 wherein the R⁺ is a phosphonium cation.

20. The ionic liquid of claim 15 wherein Rₐ, and R_{b} are independently, the same or different, H, a halo group, a C₁-C₈ alkyl group, a C₂-C₈ alkenyl group, C₂-C₈ alkynyl group, —Si(R_{d}RₑR_{f}), —(R_{g})Si(R_{d}RₑR_{f}), wherein R_{g} is a C₂-C₆ alkylene group, and R_{d}, Rₑ and R_{f} are independently a C₁-C₈ alkyl group or a C₁-C₁₀ aryl group, —(Rₕ)ORᵢ wherein Rₕ is a C₂-C₆ alkylene group and Rᵢ is a C₁-C₈ alkyl group, a C₁-C₈ haloalkyl, a C₂-C₈ alkenyl group, a phenyl group or an C₂-C₈ alkynyl group, —ORᵢ, a cyano group, a C₁-C₆ cyanoalkyl group, a C₁-C₈ hydroxyalkyl group, —(Rⱼ)(O)ORₖ, wherein Rⱼ is an C₂-C₆ alkylene group and Rₖ is a C₁-C₈ alkyl group, a C₁-C₈ haloalkyl group, a C₂-C₈ alkenyl group or a C₂-C₈ alkynyl group, an amine group, a C₁-C₈ alkylamine, a C₁-C₈ dialkylamine group, a C₁-C₈ haloalkyl group, a C₆-C₁₀ aryl group, a C₈-C₁₆ alkylaryl group, a C₈-C₁₆ arylalkyl group, —(R₁₅)ₙ[Si(Rₗ)(Rₘ)O]ₚSi(R_{q}RᵣRₛ), wherein n is an integer from 1 to 5, p is an integer from 1 to 9, wherein R₁₅ is a C₂-C₃ alkylene group, and Rₗ, Rₘ, R_{q}, Rᵣ and Rₛ are independently, the same or different, H, a C₁-C₈ alkyl group or a phenyl group, or —R₁₆O[C(R₁₇)(R₁₈)]ᵤO[C(R₁₉)(R₂₀)]ᵥO[C(R₂₁)(R₂₂)]ₓOR₂₃, wherein R₁₆ is a C₂-C₆ alkylene group, R₁₇, R₁₈, R₁₉, R₂₀ R₂₁, R₂₂ and R₂₃ are independently, the same or different, H, a C₁-C₈ alkyl group or a phenyl group, wherein u is an integer of 2 or 3, v is an integer of 0, 2 or 3 and x is an integer of 0, 2 or 3, and wherein at least one of Rₐ and R_{b} is not H.

21. The ionic liquid of claim 20 wherein neither Rₐ, and R_{b} are H.

22. The ionic liquid of claim 20 wherein Rₐ, and R_{b} are independently H, Cl, F, a C₁-C₆ alkyl group, —CF₃, —COOCH₃, —CH₂OH, —CH₂OCH₃, —C≡N, —CH₂C≡N, —C≡CH, —CH₂C≡CH, —CHC═CH₂, a phenyl group.

23. The ionic liquid of claim 22 wherein neither Rₐ, and R_{b} are H.

24. The ionic liquid of claim 20 wherein Rₐ, and R_{b} are independently H or a C₁-C₃ alkyl group.
